(19) United States
(12) Patent Application Publication
(10) Pub. No.: US 2022/0409378 A1
(43) Pub. Date: Dec. 29, 2022

(54) Apparatus for an Anatomical Prosthesis of the Temporomandibular Joint

(71) Applicant: CAUDURO PARTICIPAÇÖES EIRELI, Araquari (BR)

(72) Inventors: ROBERTO SANTOS CAUDURO, Porto Alegre (BR); MIGUEL ANGELO RIBEIRO SCHEFFER, Porto Alegre (BR)

(21) Appl. No.: 17/519,672

(22) Filed: Nov. 5, 2021

(30) Foreign Application Priority Data
Jun. 28, 2021 (BR) 10 2021 012801 1

(51) Int. Cl. A61F 2/30

(52) U.S. Cl. CPC A61F 2/3099; A61F 2002/30993; A61F 2002/30332

(57) ABSTRACT

An Apparatus for an Anatomical Prosthesis of the Temporomandibular Joint which includes a polymeric centralizing device for the pyriform condyle in the concavity of the polymeric temporal section that joins the condyle to the body of the mandibular fixture, in a stable and secure way, using a frictional interference Cone-Morse-Connection, as well as a flat part in the center of the cavity of the temporal section and posterior edges of specific dimensions, which make the fixture of the junction of the mandibular section and the temporal section more secure and allows freedom of centric movement.

### THE FIELD OF THE INVENTION

This invention is a technical solution presented as a set of elements for an anatomical prosthesis developed for the region of the temporomandibular joint (TMJ), consisting of a polymeric centralizing device for the pyriform condyle in the concavity of the temporal polymeric section, as well as a flat part in the center of the cavity of the temporal section and posterior edges of specific dimensions, which make the fixture of the junction of the mandibular section and the temporal section more secure and allows freedom of centric movement.

### THE STATE OF THE ART

There are numerous models of prostheses to replace temporomandibular joints, that are available in this country and elsewhere, which are used when, among other instances, a patient has trauma sequelae, growth or development disorders, autoimmune diseases such as rheumatoid arthritis, malignant (cancerous) or benign neoplasms, syndromes or internal disorders, etc. Some examples of temporomandibular joint (TMJ) prostheses are the well-known brands: ENGIMPLAN, KLS MARTIN and BIOMET.

Despite the numerous products available, there are frequent instances where prostheses cause injuries to patients as a result of dislocation, caused by both normal usage or trauma, components of the prosthesis becoming loose or worn, mismatched mandibular movement because they can't reproduce the physiological movement of the original joint and the distension of facial muscles and tissues.

These problems require patients to be seen, in order to correct the defects resulting from the changes to the prostheses. This sometimes leads to severe damage to the temporal mandibular region and can cause permanent problems when chewing, speaking and listening, as well as aesthetic damage to the affected area.

In other words, although there are many products available, they do not precisely satisfy the physical and social rehabilitation function that the patient needs them for, and only a small number of prostheses provide credibly similar design and function as this region of the human body.

To take an example, we can see from the State of the Art declaration in invention U.S. Pat. No. 5,549,680A, entitled Apparatus For Total Temporomandibular Joint Replacement, owned by Zimmer Biomet CMF and Thoracic LLC., that there is no centralizing device to help guide the surgeon in perfectly adjusting the condyle in the concave fossa of the polymeric temporal section or any of the other technical solutions outlined below as part of this inventive system.

A key factor for the success of the procedure in the short and long term is the ability to perfectly adjust it using the centralizing device, as inaccurate adjustment is the main reason for complaints from patients who have to have further surgery for it to be adjusted again or even have the prosthesis completely replaced because it stops working.

In the type of product based on this invention, the aim is to reproduce physiological movement, called freedom of centric movement, by having it perfectly adjusted, which is not provided by any other product on the market.

This way, physiological movement can be returned, which causes fewer dislocations, provides more stability and the joint is likely to be successful over the longer term. The combination of features in this invention, which will be described, aims to make it comfortable, return function and make it less likely to require further attention.

### DESCRIPTION OF THE INVENTION

The innovation in the invention presented here can have a significant impact on the functionality and performance of temporomandibular joint prostheses. It aims to enable patients to be able to function socially and at work as before and restore their quality of life by providing prostheses that imitate, as closely as possible, the original physiological function, with the least possible requirement for further surgery.

The difference that it makes is mainly due to the inclusion of an innovative part that allows the prosthesis to mimic the original temporomandibular joint as closely as possible, and this part is described below.

The novel feature of this prosthetic system is mainly due to an original part, the centralizing device, which enables the condyle to be centralized in the concave fossa of the polymeric temporal section, which after being used to centralize and fix the device perfectly, is removed and discarded at the end of the surgery.

The centralizing device is provided with the prosthesis, already installed in the condyle of the mandibular section. This device helps align the passive positioning of this section (or the apparatus) at the center of the glenoid fossa cavity of the polymeric temporal section of the prosthesis.

The device, already installed, helps to guide the surgeon so that the mandibular section is fixed to the mandible during surgery enabling it to be fitted to the temporal section perfectly.

Also, within the apparatus of this invention there is a flat part in the center of the cavity of the glenoid fossa of the entirely polymeric temporal section, which allows physiological movement to be reproduced, referred to as freedom of centric movement. Because such physiological movement is possible, there is less likelihood of dislocation, it provides more stability and the joint is likely to be successful over the longer term.

It includes a condyle with a pyriform design that is joined to the body of the mandibular fixture, in a stable and secure way, using a frictional interference Cone-Morse-Connection, making the apparatus more stable.

The design also features posterior edges of specific dimensions. In particular, the width and height are greater than others on the market. These differentiated edges act as convenient limits on any extreme movement by the condyle, which further improves the freedom of centric movement allowed, by installing it using the centralizing device and avoids any damage from contact with the edges.

The features of the invention mentioned above provide technical incremental improvements in comfort, the ability to restore function and reduce the chance of complications and the need for further surgery. This all means savings in time and resources over the long term due to the greater durability of the prosthesis.

### A BRIEF DESCRIPTION OF THE FIGURES

This invention can be easily explained through the detailed description provided below which illustrates, but does not limit the features of the invention, as follows:

FIG. 1 represents a complete rear view of the prosthesis, showing the temporal section (1), the centralizing device (2), the pyriform condyle (3), and the mandibular section (4);

FIG. 2 represents a complete front view of the prosthesis, showing the temporal section (1), the centralizing device (2) and the mandibular section (4);

FIG. 3 represents a complete side view of the prosthesis, showing the temporal section (1), the centralizing device (2), the pyriform condyle (3), and the mandibular section (4);

FIG. 4 represents a complete view of the underside of the prosthesis, showing the temporal section (1), the centralizing device (2), the pyriform condyle (3), and the mandibular section (4);

FIG. 5 represents a split rear view of the prosthesis, showing the temporal section (1), the centralizing device (2), the pyriform condyle (3), and the mandibular section (4);

FIG. 6 represents a split side view of the prosthesis, showing the temporal section (1), the centralizing device (2), the pyriform condyle (3), and the mandibular section (4);

FIG. 7 represents a partial rear view of the prosthesis, showing the centralizing device (2);

FIG. 8 represents a partial front view of the prosthesis, showing the centralizing device (2);

FIG. 9 represents a partial side view of the prosthesis, showing the centralizing device (2);

FIG. 10 represents a partial overhead view of the prosthesis, showing the centralizing device (2);

FIG. 11 represents a partial side view of the prosthesis, the centralizing device (2), the pyriform condyle (3), and the mandibular section (4);

FIG. 12 represents a partial overhead view of the prosthesis, the centralizing device (2), the pyriform condyle (3), and the mandibular section (4);

FIG. 13 represents a partial view of the underside of the prosthesis, showing the temporal section (1), and the flat section (5);

FIG. 14 represents a partial view of the underside of the prosthesis, showing the temporal section (1) and the differentiated posterior edges (6).

FIG. 15 represents a partial front view of the prosthesis, showing the temporal section (1) and the differentiated posterior edges (6).

FIG. 16 represents a partial rear view of the prosthesis, showing the temporal section (1) and the differentiated posterior edges (6).

FIG. 17 represents a partial side view, in section, of the prosthesis, showing the temporal section (1), the pyriform condyle (3), and the frictional interference cone-morse-connection (7);

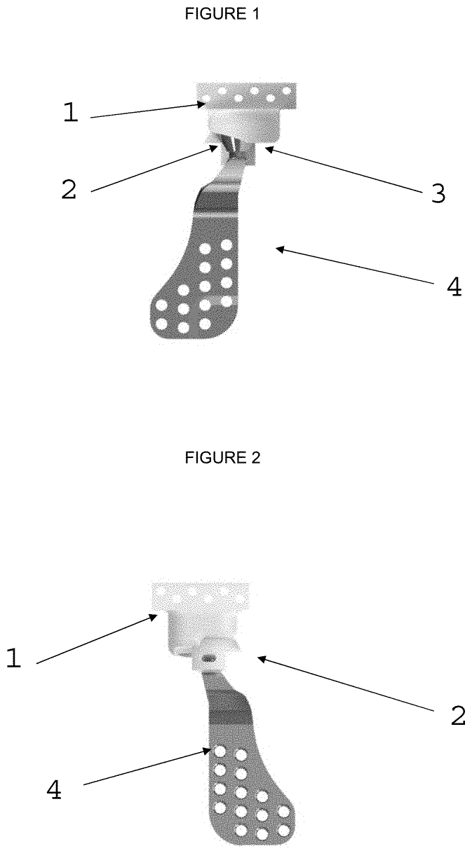

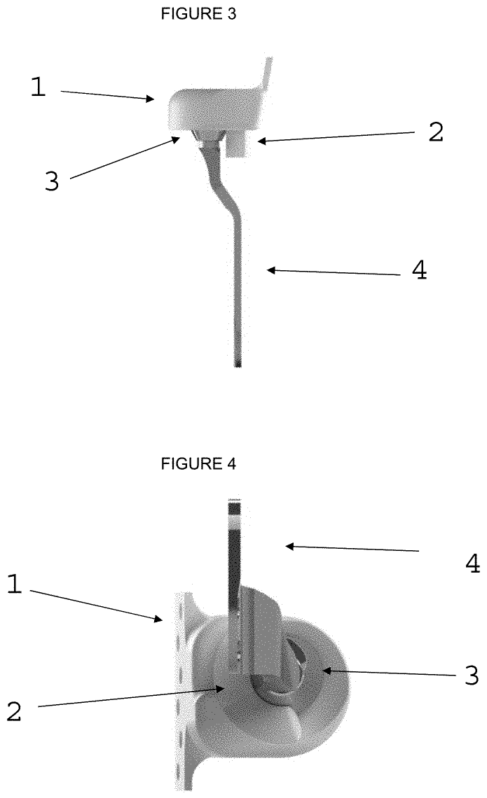

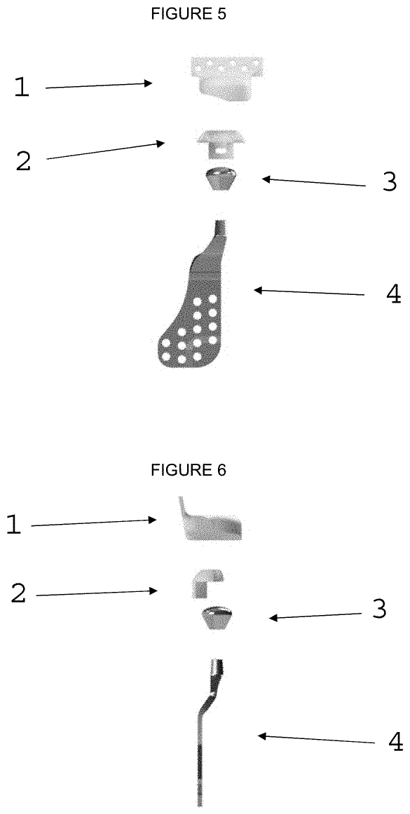

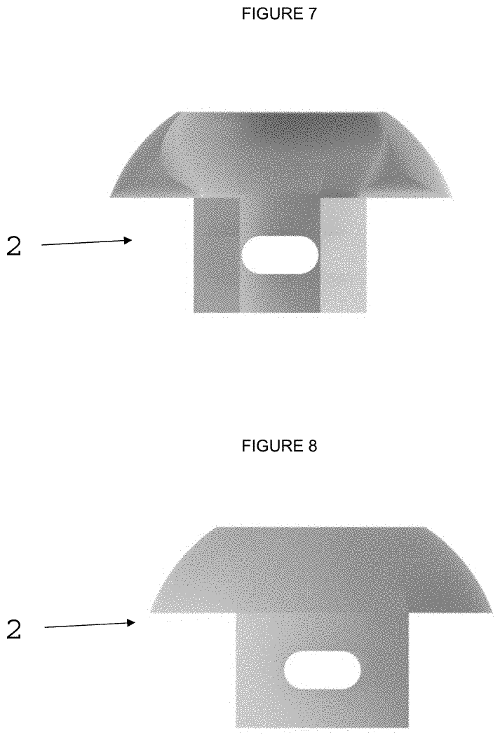

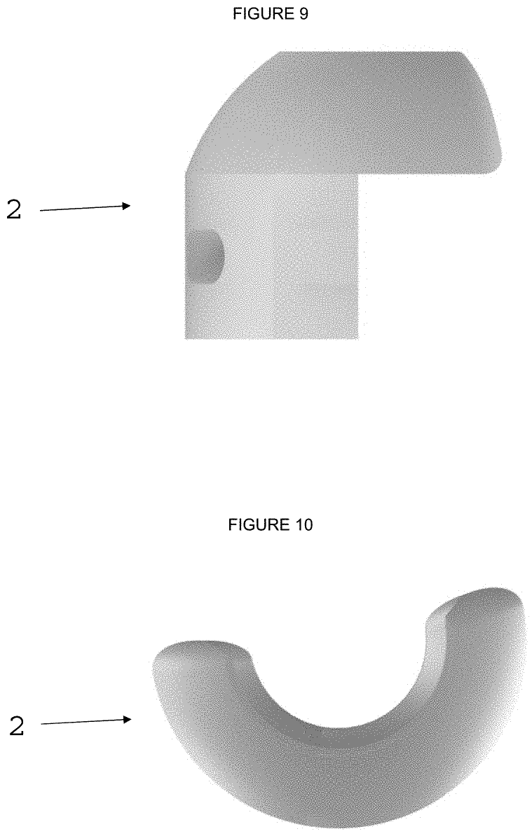

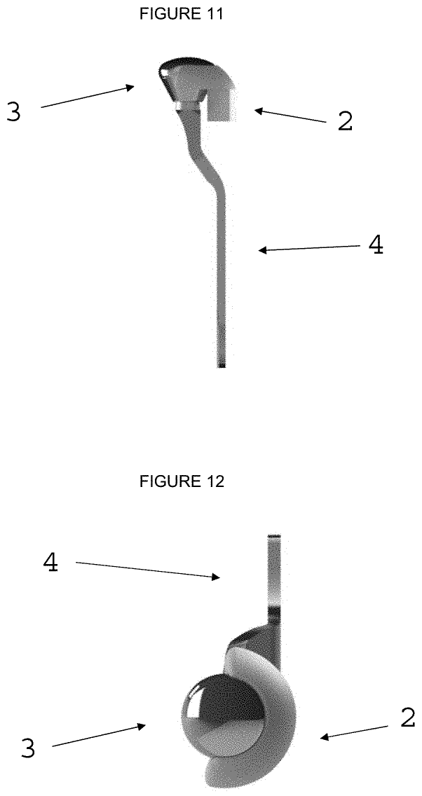

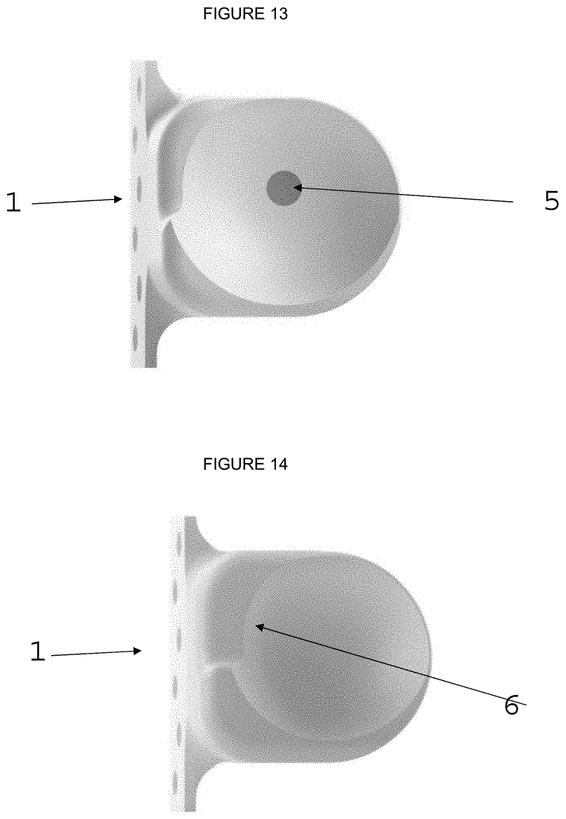

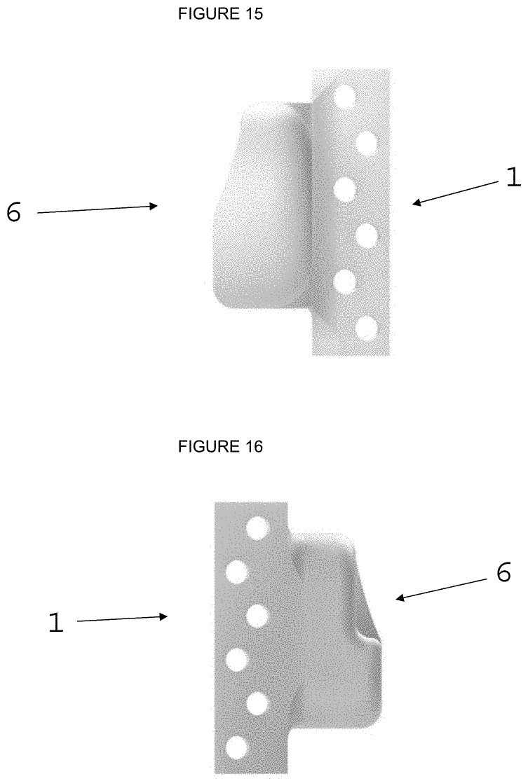

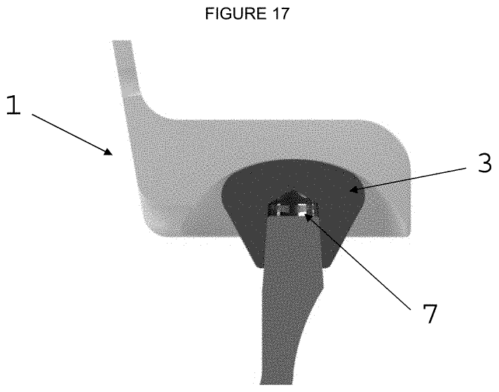

### A DETAILED DESCRIPTION OF THE INVENTION

In order for the features of the system that make up this invention to operate perfectly, the adjustments detailed below need to be made.

The novel feature of this prosthetic system is mainly due to an original device, which consists of a polymeric part that enables the condyle to be centralized in the concave fossa of the polymeric temporal section. This original device is flexible and is removed once the mandibular section of the TMJ prosthesis has been fixed in place. It consists of a semi-ring, self-locking part, with a flap, that is designed to be removed and disposed of at the end of the surgical procedure.

The centralizing device provides assistance in an innovative and original way to adjusting the centering of the condyle in the concave cavity of the temporal section when surgically installing the TMJ prosthesis.

The main advantage of this removable tool, which comes attached to the prosthesis is that, unlike all other types of TMJ prosthesis, you can be certain that the condyle has been centralized. This means that the prosthetic apparatus is unlikely to operate inconsistently or poorly, it makes physiological movement better and makes the system more predictable and extends its service life.

The polymeric temporal section has a lower surface with a semi-elliptical concave fossa, which has a radius greater than the radius of the condyle of the mandibular section, to an order of at least 7% to 30%. This allows freedom of rotation and allows the condyle to make excursive movements within the temporal fossa, without dislocating the joint on the lateral walls of the polymeric temporal section.

Also, an innovative feature is the flat part at the center of the convex cavity of this section, which means that the condyle of the mandibular section has freedom of centric movement that is physiological and similar to that of a natural joint.

This freedom of centric movement means that the condyle can be displaced up to a certain range, without any interference or trauma being caused by the component's micro movements colliding with the walls of the concave fossa of the temporal section, as occurs in natural joints. This capability protects the joints by avoiding stress from postural micro movements that are inherent in the human body.

The range of freedom of centric movement varies in a natural joint and can vary from one individual to another, normally from 01 to 03 millimeters across all vectors, due to the strength of the articular and periarticular tissues.

The system provides better stability through the invention used in the device that joins the condyle, with its pyriform shape, stably and securely, to the body of the mandibular fixture, made of Titanium ASTMF-136. This is the frictional interference Cone-Morse-Connection.

The pyriform shaped condyle contains a bio-inert alloy of Cobalt-Chrome ASTM-1537 which is extremely hard, in the form of a high precision, strict tolerance conical internal cavity for coupling to the outer cone of the terminal end of the mandibular fixture section, with a similar profile and tolerances to create maximum surface friction and consequent cold welding, by the interlocking of the cone interference.

The interference union of cones avoids the use of solder, screws or other threading, adhesives or any other devices that can affect the physical characteristics of the raw materials of the components or leave a risk of the part becoming loosened or becoming contaminated by the chemical agents in adhesives. It also encourages the junctions between the two bodies to be sealed, preventing any substances or bacteria into the interface between them.

A further development is the alteration of the posterior edges of the polymeric temporal section to specific dimensions, which increases the width and height, within the extremes of 1 mm (one millimeter) and 3 mm (three millimeters), which allows them to act as convenient limiters on any extreme movement by the condyle. These features were designed, therefore, to prevent dislocation or condylar escapes, a common problem with existing prostheses.

The posterior (distal) and latero-posterior edges of the fossa or concave cavity of the temporal section have unusual dimensions as mentioned above. They act as convenient limiters on any extreme movement by the condyle, especially movements towards the posterior or latero-posterior of the apparatus.

This feature of the invention was designed and developed to prevent dislocation and condylar escapes, a common problem with existing prostheses on the market.

The functional and anatomical characteristics presented in this invention are as similar as possible to the organ it replaces, and tries to reproduce, within the limits of this technique, its functional and anatomical characteristics.

This therefore, through the system of features that make up this invention, provides a way to reproduce movements that are most similar to physiological ones, referred to as freedom of centric movement and it can be adjusted perfectly, which is a feature that does not exist with any other available product on the market.

Using this, the natural movement of the temporomandibular joint (TMJ) can be restored, which is less likely to cause dislocation, provides better stability and better long-term success for the joint. It can also help avoid the need for further surgery, is safe and comfortable for the patients and has a low cost impact on the manufacture of prostheses.

1. An Apparatus for an Anatomical Prosthesis of the Temporomandibular Joint that consists of an anatomical prosthesis developed for the region of the temporomandibular joint (TMJ) comprising a polymeric centralizing device to center the condyle in the concave fossa of the polymeric temporal section.

2. An Apparatus for an Anatomical Prosthesis of the Temporomandibular Joint that consists of a condyle with a pyriform design, as mentioned in claim 1, the feature of which is that it is fixed by a frictional interference Cone-Morse-Connection.

3. An Apparatus for an Anatomical Prosthesis of the Temporomandibular Joint comprising an anatomical prosthesis for the region of the temporomandibular joint (TMJ), which features a flat part in the center of the cavity of the glenoid fossa of the temporal section.

4. An Apparatus for an Anatomical Prosthesis of the Temporomandibular Joint comprising an anatomical prosthesis for the region of the temporomandibular joint (TMJ), which features posterior edges with specific dimensions for the polymeric temporal section.
